(19) United States
(12) Patent Application Publication
(10) Pub. No.: US 2024/0269428 A1
(43) Pub. Date: Aug. 15, 2024

(54) MEDICAL DEVICE FOR ULTRASOUND-ASSISTED DRUG DELIVERY

(71) Applicant: Boston Scientific Scimed, Inc., Maple Grove, MN (US)

(72) Inventors: Benjamin Montag, Delano, MN (US); Curtis Cornell Genstler, Snohomish, WA (US); Joseph Czyscon, Elk River, MN (US)

(73) Assignee: Boston Scientific Scimed, Inc., Maple Grove, MN (US)

(21) Appl. No.: 18/438,738

(22) Filed: Feb. 12, 2024

Related U.S. Application Data
(60) Provisional application No. 63/445,184, filed on Feb. 13, 2023.

(51) Int. Cl. A61M 25/00; A61B 17/22

(52) U.S. Cl. CPC A61M 25/005; A61B 17/2202

(57) ABSTRACT

A system for treating a vascular region includes an elongate catheter shaft having a proximal region and a distal region. The elongate catheter shaft includes an inner lumen extending through the elongate catheter shaft, the inner lumen adapted to allow a cooling media to pass therethrough, a plurality of cooling media ports in fluid communication with the inner lumen, a drug delivery lumen extending adjacent to the inner lumen, and a plurality of drug delivery ports in fluid communication with the drug delivery lumen. The system includes an ultrasound catheter adapted to be disposed within the inner lumen, the ultrasound catheter including a plurality of ultrasound transducers.

### CROSS-REFERENCE TO RELATED APPLICATIONS

This application claims the benefit of priority under 35 U.S.C. § 119 of U.S. Provisional Application No. 63/445,184, filed Feb. 13, 2023, the entire disclosure of which is hereby incorporated by reference.

### TECHNICAL FIELD

The present disclosure pertains to elongated intracorporeal medical devices. More particularly, the present disclosure pertains to elongated intracorporeal medical devices that include ultrasound transducers.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices. Moreover, there is a need for medical devices that can provide constructive interference within ultrasound fields provided by ultrasound transducers in combination with introduction of lytic drugs.

### SUMMARY

This disclosure provides design, material, manufacturing method, and use alternatives for medical devices. An example may be found in a system for treating a vascular region. The system includes an elongate catheter shaft having a proximal region and a distal region. The elongate catheter shaft includes an inner lumen extending through the elongate catheter shaft that is adapted to allow a cooling media to pass therethrough, a plurality of cooling media ports in fluid communication with the inner lumen, a drug delivery lumen extending adjacent to the inner lumen, and a plurality of drug delivery ports in fluid communication with the drug delivery lumen. The system includes an ultrasound catheter that adapted to be disposed within the inner lumen and that includes a plurality of ultrasound transducers.

Alternatively or additionally, the inner lumen may be adapted to allow for the cooling media to pass therethrough when the ultrasound catheter is disposed within the inner lumen.

Alternatively or additionally, at least some of the plurality of cooling media ports may be disposed within the distal region of the elongate catheter shaft.

Alternatively or additionally, at least some of the plurality of cooling media ports may extend through a side wall of the elongate catheter shaft.

Alternatively or additionally, at least some of the plurality of cooling media ports may be axially spaced from neighboring cooling media ports.

Alternatively or additionally, at least some of the plurality of cooling media ports may axially align relative to at least some of the plurality of ultrasound transducers when the ultrasound catheter is disposed within the inner lumen.

Alternatively or additionally, at least some of the plurality of cooling media ports may be arranged in a spiral configuration in which each cooling media port is axially spaced and circumferentially spaced from neighboring cooling media ports.

Alternatively or additionally, at least some of the plurality of drug delivery ports may be disposed within the distal region of the elongate catheter shaft.

Alternatively or additionally, at least some of the plurality of drug delivery ports may extend through a side wall of the elongate catheter shaft.

Alternatively or additionally, at least some of the plurality of drug delivery ports may be arranged in a spiral configuration in which each drug delivery port is axially spaced and circumferentially spaced from neighboring drug delivery ports.

Alternatively or additionally, each drug delivery port may be axially and/or circumferentially spaced from neighboring cooling media ports.

Alternatively or additionally, the system may further include a terminal cooling media port disposed at a distal end of the elongate catheter shaft, the elongate catheter shaft and the ultrasound catheter adapted to selectively open or close the terminal cooling media port.

Another example may be found in a system for treating a vascular region. The system includes an ultrasound catheter including a plurality of ultrasound transducers. An elongate catheter shaft defines a central lumen and a plurality of peripheral lumens extending through the elongate catheter shaft, the central lumen adapted to accommodate the ultrasound catheter extending therethrough, the elongate catheter shaft including a side wall. A plurality of central lumen ports extend through the side wall and are in fluid communication with the central lumen.

A plurality of peripheral lumen ports extend through the side wall and are in fluid communication with the plurality of peripheral lumens.

Alternatively or additionally, at least some of the plurality of central lumen ports may be axially spaced from neighboring central lumen ports.

Alternatively or additionally, at least some of the plurality of central lumen ports may axially align relative to at least some of the plurality of ultrasound transducers when the ultrasound catheter is disposed within the central lumen.

Alternatively or additionally, at least some of the plurality of central lumen ports may be arranged in a spiral configuration in which each central lumen port is axially spaced and circumferentially spaced from neighboring central lumen ports.

Alternatively or additionally, at least some of the plurality of peripheral lumen ports may be arranged in a spiral configuration in which each peripheral lumen port is axially spaced and circumferentially spaced from neighboring peripheral lumen ports.

Alternatively or additionally, each central lumen port may be axially and/or circumferentially spaced from neighboring peripheral lumen ports.

Another example may be found in a system for treating a vascular region. The system includes an ultrasound catheter including a plurality of ultrasound transducers and an elongate catheter shaft defining an inner lumen and a plurality of outer lumens extending through the elongate catheter shaft, the inner lumen adapted to accommodate the ultrasound catheter extending therethrough, the elongate catheter shaft including a treatment zone that corresponds to where the plurality of ultrasound transducers are when the ultrasound catheter is disposed within the inner lumen. A plurality of inner lumen ports extend through the side wall and are in fluid communication with the inner lumen. A plurality of outer lumen ports extend through the side wall and are in fluid communication with the plurality of outer lumens.

Alternatively or additionally, at least some of the plurality of inner lumen ports and at least some of the plurality of outer lumen ports may be disposed within the treatment zone.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a schematic view of certain features of an illustrative catheter;

FIG. 2 is a cross-sectional view taken along line 2-2 of FIG. 1;

FIG. 3 is a schematic view of an illustrative elongate inner core configured to be positioned within the central lumen of the catheter shown in FIG. 2;

FIG. 4 is a cross-sectional view taken along line 4-4 of FIG. 3;

FIG. 5 is a schematic view of an illustrative elongate treatment core.

FIG. 6 is a schematic view of an illustrative catheter;

FIG. 6A is a schematic view of a portion of the illustrative catheter of FIG. 5;

FIG. 7 is a cross-sectional view taken along the line 7-7 of FIG. 6;

FIG. 8 is a cross-sectional view taken along the line 8-8 of FIG. 6;

FIG. 9 is a cross-sectional view taken along the line 9-9 of FIG. 6;

FIG. 10 is a cross-sectional view taken along the line 10-10 of FIG. 6;

FIG. 11 is a cross-sectional view taken along the line 11-11 of FIG. 6;

FIG. 12 is a cross-sectional view taken along the line 12-12 of FIG. 6;

FIG. 13 is a schematic view of an illustrative catheter;

FIGS. 14A and 14B together provide an illustrative example of closing a terminal cooling media port; and

FIGS. 15A and 15B together provide an illustrative example of closing a terminal cooling media port.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result).

In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used in connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

As used herein, the term “ultrasonic energy” is used broadly, includes its ordinary meaning, and further includes mechanical energy transferred through pressure or compression waves with a frequency greater than about 20 kHz. Ultrasonic energy waves have a frequency between about 500 kHz and about 20 MHz in one example embodiment, between about 1 MHz and about 3 MHz in another example embodiment, of about 3 MHz in another example embodiment, and of about 2 MHz in another example embodiment. As used herein, the term “catheter” is used broadly, includes its ordinary meaning, and further includes an elongate flexible tube configured to be inserted into the body of a patient, such as into a body part, cavity, duct or vessel. As used herein, the term “therapeutic compound” is used broadly, includes its ordinary meaning, and encompasses drugs, medicaments, dissolution compounds, genetic materials, microbubbles, nanobubbles, nanoparticles or phase-shift nanodroplets, and other substances capable of effecting physiological functions through either chemical reaction with substances within the body or through physical interaction with tissue in the body. A mixture comprising such substances is encompassed within this definition of “therapeutic compound”. As used herein, the term “end” is used broadly, includes its ordinary meaning, and further encompasses a region generally, such that “proximal end” includes “proximal region”, and “distal end” includes “distal region”.

As expounded herein, ultrasonic energy is often used to enhance the delivery and/or effect of a therapeutic compound. For example, in the context of treating vascular occlusions, ultrasonic energy has been shown to increase enzyme mediated thrombolysis by enhancing the delivery of thrombolytic agents into a thrombus, where such agents lyse the thrombus by degrading the fibrin that forms the thrombus. The thrombolytic activity of the agent is enhanced in the presence of ultrasonic energy in the thrombus. However, it should be appreciated that the invention should not be limited to the mechanism by which the ultrasound enhances treatment unless otherwise stated. In other applications, ultrasonic energy has also been shown to enhance transfection of gene-based drugs into cells, and augment transfer of chemotherapeutic drugs into tumor cells. Ultrasonic energy delivered from within a patient's body has been found to be capable of producing non-thermal effects that increase biological tissue permeability to therapeutic compounds by up to or greater than an order of magnitude.

Use of an ultrasound catheter to deliver ultrasonic energy and a therapeutic compound directly to the treatment site mediates or overcomes many of the disadvantages associated with systemic drug delivery, such as low efficiency, high therapeutic compound use rates, and significant side effects caused by high doses. Local therapeutic compound delivery has been found to be particularly advantageous in the context of thrombolytic therapy, chemotherapy, radiation therapy, and gene therapy, as well as in applications calling for the delivery of proteins and/or therapeutic humanized antibodies. However, it should be appreciated that in certain arrangements the ultrasound catheter can also be used in combination with systemic drug delivery instead or in addition to local drug delivery. In addition, local drug delivery can be accomplished through the use of a separate device (e.g., catheter).

As will be described below, the ultrasound catheter can include two or more ultrasound radiating members positioned therein. Such ultrasound radiating members can include a transducer (e.g., a PZT transducer), which is configured to convert electrical energy into ultrasonic energy. In such embodiments, the PZT transducer is excited by specific electrical parameters (herein “power parameters” that cause it to vibrate in a way that generates ultrasonic energy).

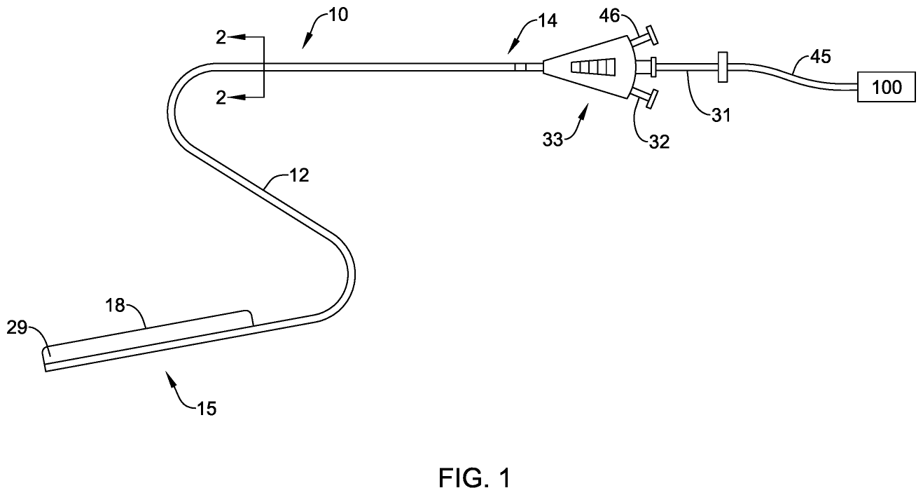

With reference to the illustrated embodiments, FIG. 1 illustrates an ultrasonic catheter 10 configured for use in a patient's vasculature. For example, in certain applications the ultrasonic catheter 10 is used to treat long segment peripheral arterial occlusions, such as those in the vascular system of the leg, while in other applications the ultrasonic catheter 10 is used to treat occlusions in the small vessels of the neurovasculature or other portions of the body (e.g., other distal portions of the vascular system). The ultrasonic catheter 10 may be used to treat PE (pulmonary embolisms) and DVT (deep vein thrombosis), for example. Thus, the dimensions of the catheter 10 may be adjusted based on the particular application for which the catheter 10 is to be used.

The ultrasonic catheter 10 generally includes a multi-component, elongate shaft 12 having a proximal region 14 and a distal region 15. The elongate shaft includes a flexible energy delivery section 18 located in the distal region 15 of the catheter 10. In some instances, the energy delivery section 18 may be considered as being a treatment zone, for example. The elongate shaft 12 and other components of the catheter 10 are manufactured in accordance with a variety of techniques. Suitable materials and dimensions are selected based on the natural and anatomical dimensions of the treatment site and on the desired percutaneous access site.

For example, in an embodiment the proximal region 14 of the elongate shaft 12 may include a material that has sufficient flexibility, kink resistance, rigidity and structural support to push the energy delivery section 18 through the patient's vasculature to a treatment site. Examples of such materials include, but are not limited to, extruded polytetrafluoroethylene (“PTFE”), polyethylenes (“PE”), polyamides and other similar materials. In certain embodiments, the proximal region 14 of the elongate shaft 12 may be reinforced by braiding, mesh or other constructions to provide increased kink resistance and pushability. For example, in certain embodiments nickel titanium or stainless steel wires may be placed along or incorporated into the elongate shaft 12 to reduce kinking.

In some instances, the energy delivery section 18 of the elongate shaft 12 may be formed of a material that (a) is thinner than the material forming the proximal region 14 of the elongate shaft 12, or (b) has a greater acoustic transparency than the material forming the proximal region 14 of the elongate shaft 12. Thinner materials generally have greater acoustic transparency than thicker materials. Suitable materials for the energy delivery section 18 include, but are not limited to, high or low density polyethylenes, urethanes, nylons, and the like. In some embodiments, the energy delivery section 18 is formed from the same material or a material of the same thickness as the proximal region 14.

One or more fluid delivery lumens may be incorporated into the elongate shaft 12. For example, in one embodiment a central lumen passes through the elongate shaft 12. The central lumen extends through the length of the elongate shaft 12, and is coupled to a distal exit port 29 and a proximal access port 31. The proximal access port 31 forms part of a hub 33, which is attached to the proximal region 14 of the catheter 10. In some cases, the hub 33 may include a cooling fluid fitting 46, which is hydraulically connected to a lumen within the tubular body 12. In some cases, the hub 33 may also include a therapeutic compound inlet port 32, which is hydraulically connected to a lumen within the tubular body 12. In some cases, the therapeutic compound inlet port 32 may also be hydraulically coupled to a source of therapeutic compound via a hub such as a Luer fitting.

The catheter 10 is configured to have two or more ultrasound radiating members positioned therein. For example, in certain embodiments an ultrasound radiating member may be fixed within the energy delivery section 18 of the elongate shaft 12, while in other embodiments a plurality of ultrasound radiating members are fixed to an assembly that is passed into the central lumen. In either case, the one or more ultrasound radiating members are electrically coupled to a control system 100 via a cable 45. In one embodiment, the outer surface of the energy delivery section 18 can include a cavitation promoting surface configured to enhance/promote cavitation at the treatment site. In some cases, a cavitation promoting surface is a textured surface that can retain small pockets of air when submerged. The small pockets of air can server as a source for microbubbles or nanobubbles, thereby reducing the threshold for cavitation in an ultrasound field. In some cases, the outer surface of the energy delivery section 18 may be coated with a coating that includes components that will lower the cavitation threshold. As an example, the surface may be hydrophobic and textured in a way so that the textured surface presents a lower cavitation threshold than the surrounding bulk fluid. This can enhance the therapeutic effect of the ultrasound.

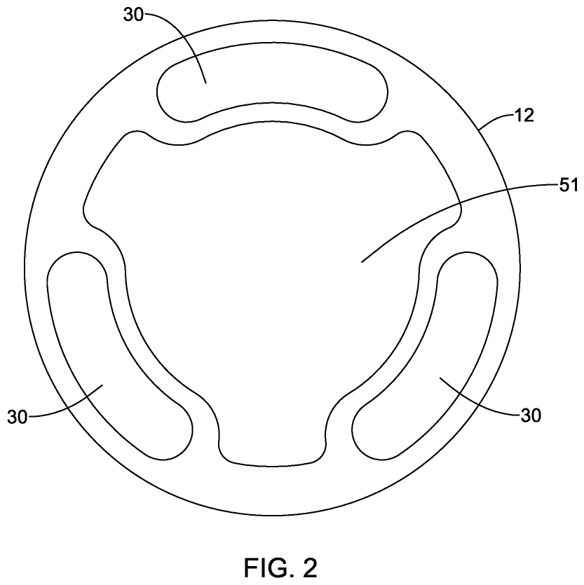

FIG. 2 illustrates a cross section of the elongate shaft 12 taken along line 2-2 of FIG. 1. As shown in FIG. 2, three fluid delivery lumens 30 may be incorporated into the elongate shaft 12. In other embodiments, more or fewer fluid delivery lumens can be incorporated into the elongate shaft 12. The elongate shaft 12 may include a hollow central lumen 51 passing through the elongate shaft 12. The cross-section of the elongate shaft 12, as illustrated in FIG. 2, may be substantially constant along most of the length of the catheter 10. Thus, in such embodiments, substantially the same cross-section is present in both the proximal region 14 and the distal region 15 of the catheter 10. In some cases, the cross-section may vary within the energy delivery section 18. In some cases, the central lumen 51 may be adapted to accommodate a coolant provided through the central lumen, even when a treatment core is present within the central lumen 51.

In certain embodiments, the central lumen 51 has a minimum diameter greater than about 0.030 inches. In another embodiment, the central lumen 51 has a minimum diameter greater than about 0.037 inches. In another embodiment, the fluid delivery lumens 30 have dimensions of about 0.026 inches wide by about 0.0075 inches high, although other dimensions may be used in other applications.

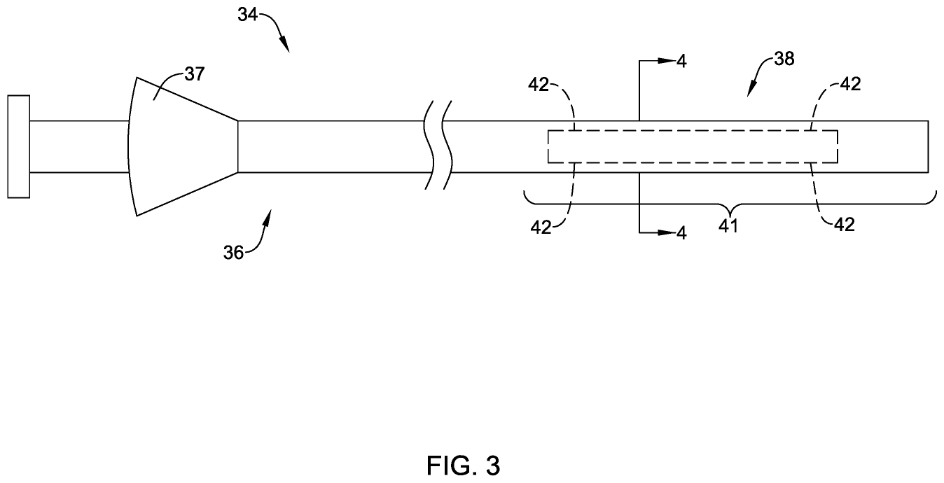

As described above, the central lumen 51 may extend through the length of the elongate shaft 12. As shown in FIG. 1, the central lumen 51 includes a distal exit port 29 and a proximal access port 31. The proximal access port 31 forms part of the hub 33, which is attached to the proximal region 14 of the catheter 10. The central lumen 51 may be configured to receive an elongate inner core 34 of which an embodiment is illustrated in FIG. 3. In some cases, the elongate inner core 34 includes a proximal region 36 and a distal region 38. A proximal hub 37 is fitted on the inner core 34 at one end of the proximal region 36. One or more ultrasound radiating members are positioned within an inner core energy delivery section 41 located within the distal region 38. The ultrasound radiating members form an ultrasound assembly 42, which will be described in detail below.

As used herein, the terms “ultrasonic energy”, “ultrasound” and “ultrasonic” are broad terms, having their ordinary meanings, and further refer to, without limitation, mechanical energy transferred through longitudinal pressure or compression waves. Ultrasonic energy can be emitted as continuous or pulsed waves, depending on the requirements of a particular application. Additionally, ultrasonic energy can be emitted in waveforms having various shapes, such as sinusoidal waves, triangle waves, square waves, or other wave forms. Ultrasonic energy includes sound waves. In certain embodiments, the ultrasonic energy has a frequency between about 20 kHz and about 20 MHz. For example, in one embodiment, the waves have a frequency between about 500 kHz and about 20 MHz. In another embodiment, the waves have a frequency between about 1 MHz and about 3 MHz. In yet another embodiment, the waves have a frequency of about 2 MHz. The average acoustic power for each ultrasound radiating member is between about 0.01 watts and 300 watts. In some embodiments, the average acoustic power for each ultrasound radiating member is about 0.2 watts and about 2.5 watts. In an embodiment, the average acoustic power for each ultrasound radiating member is about 0.27 watts.

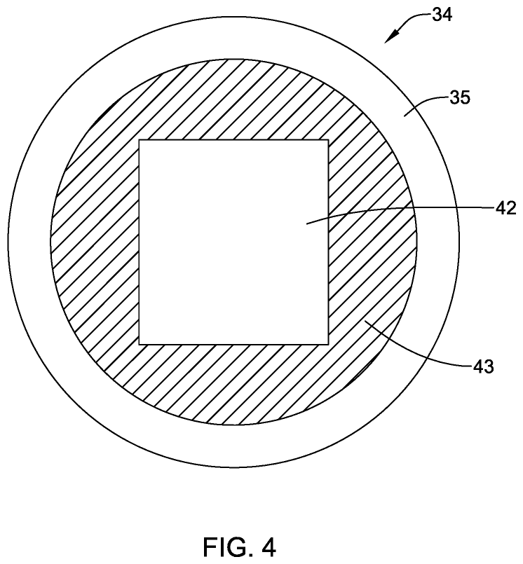

As shown in the cross-section illustrated in FIG. 4, which is taken along the line 4-4 of FIG. 3, the inner core 34 may have a cylindrical shape, with an outer diameter that permits the inner core 34 to be inserted into the central lumen 51 of the tubular body 12 via the proximal access port 31. Suitable outer diameters of the inner core 34 include, but are not limited to, about 0.010 inches to about 0.100 inches. In another embodiment, the outer diameter of the inner core 34 is between about 0.020 inches and about 0.080 inches. In yet another embodiment, the inner core 34 has an outer diameter of about 0.035 inches.

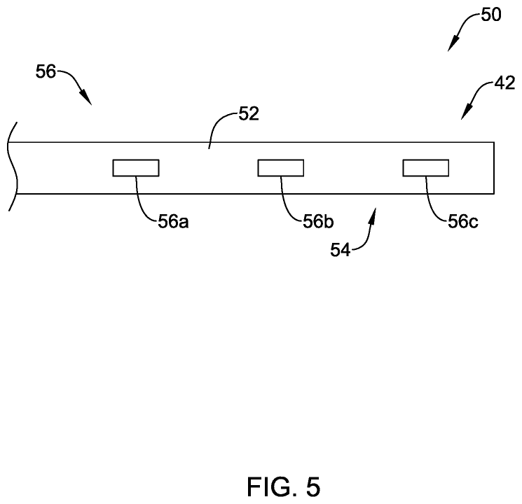

Still referring to FIG. 4, the inner core 34 may include a cylindrical outer body 35 that houses the ultrasound assembly 42. The ultrasound assembly 42 includes wiring and ultrasound radiating members, described in greater detail in FIG. 5, such that the ultrasound assembly 42 is capable of radiating ultrasonic energy from the energy delivery section 41 of the inner core 34. The ultrasound assembly 42 is electrically connected to the hub 33, where the inner core 34 can be connected to a control system 100 via cable 45 (illustrated in FIG. 1). In some cases, an electrically insulating potting material 43 fills the inner core 34, surrounding the ultrasound assembly 42, thus preventing movement of the ultrasound assembly 42 with respect to the outer body 35. In one embodiment, the thickness of the outer body 35 is between about 0.0002 inches and 0.010 inches. In another embodiment, the thickness of the outer body 35 is between about 0.0002 inches and 0.005 inches. In yet another embodiment, the thickness of the outer body 35 is about 0.0005 inches.

As used herein, the term “ultrasound radiating member” refers to any apparatus capable of producing ultrasonic energy. For example, in one embodiment, an ultrasound radiating member comprises an ultrasonic transducer, which converts electrical energy into ultrasonic energy. A suitable example of an ultrasonic transducer for generating ultrasonic energy from electrical energy includes, but is not limited to, piezoelectric ceramic oscillators. Piezoelectric ceramics may include a crystalline material, such as quartz, that changes shape when an electrical voltage is applied to the material. This change in shape, made oscillatory by an oscillating driving signal, creates ultrasonic sound waves. In other embodiments, ultrasonic energy can be generated by an ultrasonic transducer that is remote from the ultrasound radiating member, and the ultrasonic energy can be transmitted, via, for example, a wire that is coupled to the ultrasound radiating member.

FIG. 5 is a schematic view of an illustrative treatment core 50. In some cases, the illustrative treatment core 50 may be an ultrasound catheter, and may be considered as being an example of the elongate inner core 34 (FIG. 3). The treatment core 50 may be disposable within the flexible energy delivery section 18 of the tubular body 12. In some cases, the treatment core 50 may be considered as including an elongate shaft 52 that extends to a distal region 54. The elongate shaft 52 may be made of any suitable material, such as but not limited to polyimide, PET (polyethylene terephthalate) or epoxy, and may have a length in a range of 40 centimeters to 150 centimeters and a diameter in a range of 1 millimeter to 4 millimeter.

The treatment core 50 may include one or more ultrasound radiating members 56, individually labeled as 56a, 56b and 56c. It will be appreciated that only a small fraction of the elongate shaft 52 is visible in FIG. 5, and thus the treatment core 50 may include considerably more ultrasound radiating members 56. In some cases, the ultrasound radiating members 56 may be considered as having an axial spacing between adjacent ultrasound radiating members 56 that is in a range of 0.5 centimeters to 4 centimeters.

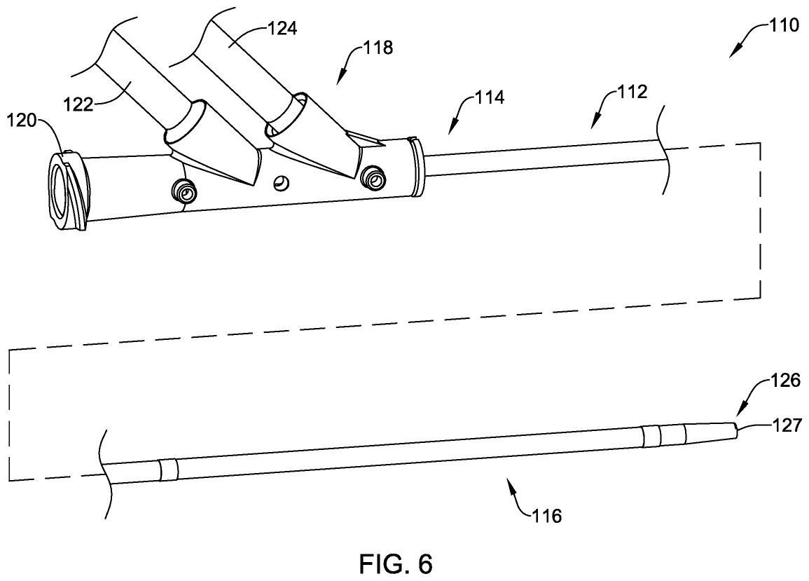

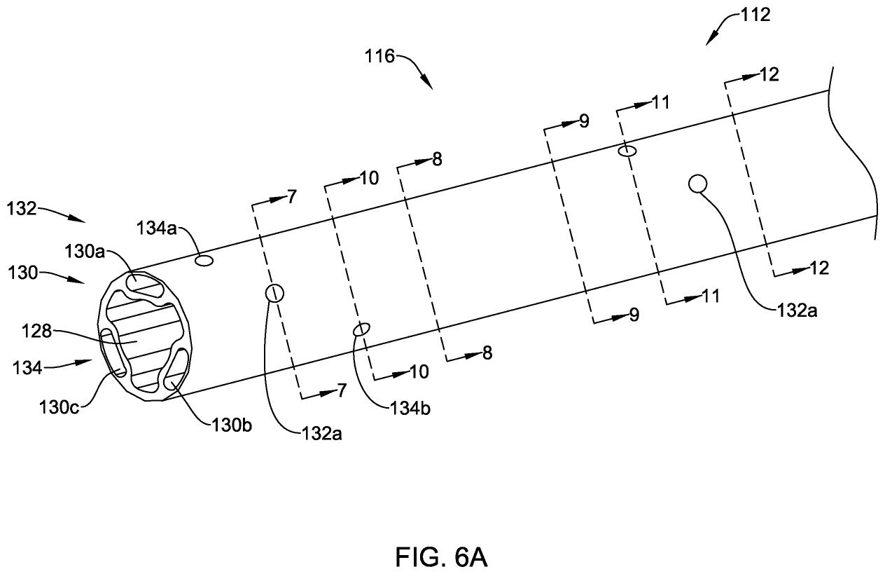

FIG. 6 is a schematic view of an illustrative catheter 110 that may be considered as being an example of the catheter 10. The illustrative catheter 110 includes an elongate shaft 112 that extends from a proximal region 114 to a distal region 116. In some instances, as shown, a proximal hub 118 may be secured relative to the proximal region 114 of the elongate shaft 112. The proximal hub 118 and the elongate shaft 112 may be adapted to permit advancing the catheter 110 over a guidewire, for example. In some instances, as shown, the proximal hub 118 may include a Luer fitting 120, a first port 122 and a second port 124 that may each be used to provide various fluids to lumens extending within the elongate shaft 112, as will be discussed. The elongate shaft 112 includes a distal tip 126. In some instances, the distal tip 126 may be an atraumatic distal tip. In some instances, the distal tip 126 may include a terminal fluid port 127 that is fluidly coupled with a lumen extending within the elongate shaft 12.

FIG. 6A is a schematic view of the distal portion 116 of the elongate shaft 112, showing several features of the elongate shaft 112. In comparison with FIG. 2, it can be seen that the elongate shaft 112 may have an internal structure that is quite similar to that of the elongate shaft 12 (FIG. 1). The elongate shaft 112 includes an inner or central lumen 128 that extends through the elongate shaft 112 and several peripheral lumens 130 that extend in parallel with each other and with the inner or central lumen 128. In some cases, the inner or central lumen 128 may accommodate an ultrasound catheter therein, and may also accommodate the flow of cooling media through the inner or central lumen 128. In some cases, the peripheral lumens 130 may accommodate the flow of therapeutic agents such as lysing drugs.

In some cases, the elongate shaft 112 includes a plurality of central lumen ports 132 that are fluidly coupled with the inner or central lumen 128 as well as a plurality of peripheral lumen ports 134 that are fluidly coupled with the peripheral lumens 130. In some instances, at least some of the central lumen ports 132 may have a round shape. In some instances, at least some of the central lumen ports 132 may have an elongated or ovoid shape. In some instances, at least some of the peripheral lumen ports 134 may have a round shape. In some instances, at least some of the peripheral lumen ports 134 may have an elongated or ovoid shape. The peripheral lumen ports 134 may be dimensioned to accommodate microbubbles, for example. As can be seen, the peripheral lumens 130 may include a peripheral lumen 130a, a peripheral lumen 130b and a peripheral lumen 130c. While a total of three peripheral lumens 130 are shown, it will be appreciated that in some cases the elongate shaft 112 may include only one or two peripheral lumens 130, or may even include four or more peripheral lumens 130.

In some instances, at least some or all of the plurality of central lumen ports 132 may be axially spaced from neighboring central lumen ports 132. In some instances, at least some or all of the plurality of central lumen ports 132 may be circumferentially spaced from neighboring central lumen ports 132. In some instances, at least some or all of the plurality of central lumen ports 132 may be considered as being arranged in a spiral, with each central lumen port 132 axially and circumferentially spaced from its neighboring central lumen ports 132. In some cases, at least some of the central lumen ports 132 may be axially spaced but not circumferentially spaced from its neighboring central lumen ports 132. In some cases, at least some of the central lumen ports 132 may be circumferentially spaced but not axially spaced from its neighboring central lumen ports 132. These are just examples, as a variety of arrangements are contemplated.

In some instances, at least some or all of the plurality of peripheral lumen ports 134 may be axially spaced from neighboring peripheral lumen ports 134. In some instances, at least some or all of the plurality of peripheral lumen ports 134 may be circumferentially spaced from neighboring peripheral lumen ports 134. In some instances, at least some or all of the plurality of peripheral lumen ports 134 may be considered as being arranged in a spiral, with each peripheral lumen ports 134 axially and circumferentially spaced from its neighboring peripheral lumen ports 134. In some cases, at least some of the peripheral lumen ports 134 may be axially spaced but not circumferentially spaced from its neighboring peripheral lumen ports 134. In some cases, at least some of the peripheral lumen ports 134 may be circumferentially spaced but not axially spaced from its neighboring peripheral lumen ports 134. In some instances, each of the central lumen ports 132 are axially and circumferentially spaced from its neighboring peripheral lumen ports 134. These are just examples, as a variety of arrangements are contemplated.

In some instances, at least some of the central lumen ports 132 may be positioned such that the central lumen ports 132 are located between the ultrasound radiating members 56 when the treatment core 50 is disposed within the inner or central lumen 128. In some instances, at least some of the peripheral lumen ports 134 may be positioned such that the peripheral lumen ports 134 are aligned with the ultrasound radiating members 56 when the treatment core 50 is disposed within the inner or central lumen 128. In some instances, at least some of the central lumen ports 132 and/or at least some of the peripheral lumen ports 134 are positioned such that the central lumen ports 132 and/or the peripheral lumen ports 134 are aligned with the ultrasound radiating members 56 when the treatment core 50 is disposed within the inner or central lumen 128. In some instances, at least some of the central lumen ports 132 and/or at least some of the peripheral lumen ports 134 are positioned such that the central lumen ports 132 and/or the peripheral lumen ports 134 are located between the ultrasound radiating members 56 when the treatment core 50 is disposed within the inner or central lumen 128.

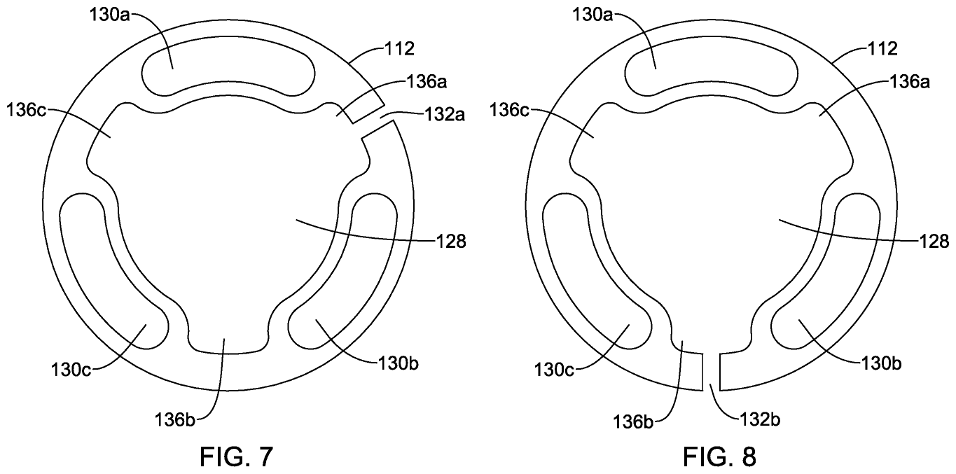

The relative arrangement of the central lumen ports 132 and the peripheral lumen ports 134 are illustrated in FIGS. 7 through 12, which are cross-sectional views through the elongate shaft 12, taken along the lines 7-7, 8-8, 9-9, 10-10, 11-11 and 12-12, respectively. FIGS. 7, 8 and 9 show the respective position of a central lumen port 132a, a central lumen port 132b and a central lumen port 132c while FIGS. 10, 11 and 12 show the respective position of a peripheral lumen port 134a, a peripheral lumen port 134b and a peripheral lumen port 134c.

In FIG. 7, it can be seen that the inner or central lumen 128 may be considered, in cross-section, as including several channels 136, individually labeled as 136a, 136b and 136c. It will be appreciated that the channels 136 are fluidly coupled with the inner or central lumen 128.

In some instances, the channels 136 may accommodate fluid flow, such as but not limited to cooling media such as saline, through the inner or central lumen 128, when another device such as the treatment core 50 is disposed within the inner or central lumen 128. The central lumen port 132a extends through the elongate shaft 112 and is fluidly coupled with the inner or central lumen 128 via the channel 136a.

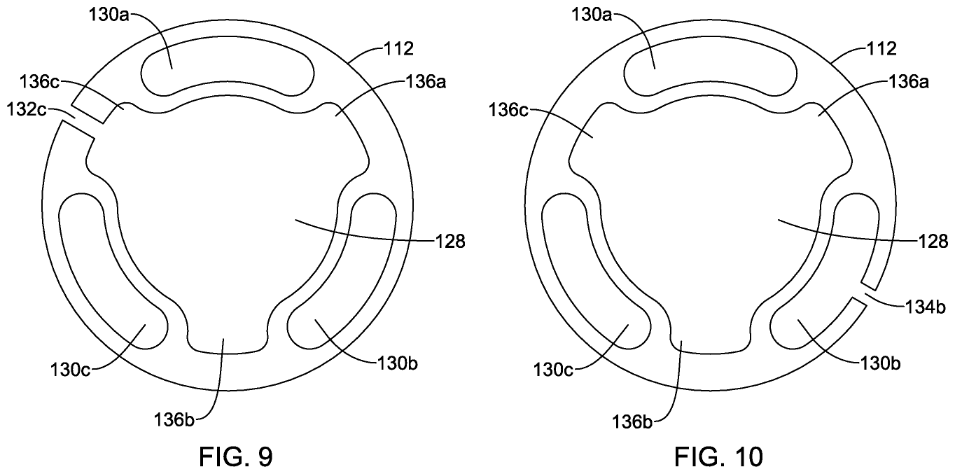

In FIG. 8, a central lumen port 132b extends through the elongate shaft 112 and is fluidly coupled with the inner or central lumen 128 via the channel 136b. In FIG. 9, a central lumen port 132c extends through the elongate shaft 112 and is fluidly coupled with the inner or central lumen 128 via the channel 136c. While only three central lumen ports 132 are called out, it will be appreciated that depending on the length of the elongate shaft 112, and the number and relative positioning of the ultrasound transducers 56 within the treatment core 50, the elongate shaft 112 may include a greater number of central lumen ports 132. It will be appreciated that the relative axial spacing of the central lumen ports 132 may be determined based upon the relative spacing between adjacent ultrasound transducers 56, for example.

Cooling media such as saline may be eluted into the vessel proximate each of the ultrasound transducers 56, for example. In some instances, at least some of the central lumen ports 132 may be axially spaced from where the ultrasound transducers 56 will be positioned when the treatment core 50 is disposed within the central or inner lumen 128, particularly if there is a desire to elute at least some of the cooling media upstream of where the ultrasound transducers 56 are located.

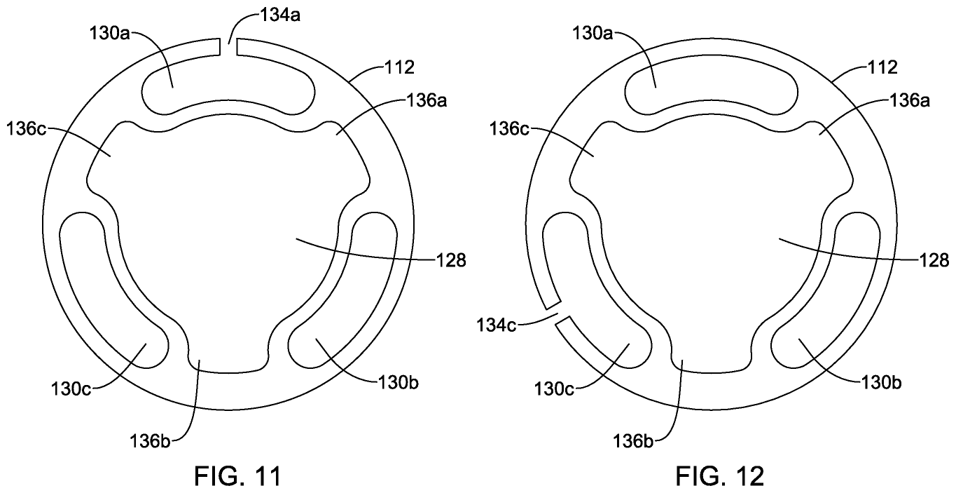

In FIG. 10, the peripheral lumen port 134b extends through the elongate shaft 112 and is fluidly coupled with the peripheral lumen 134b. In FIG. 11, the peripheral lumen port 134a extends through the elongate shaft 112 and is fluidly coupled with the peripheral lumen 134a. In FIG. 12, the peripheral lumen port 134c extends through the elongate shaft 112 and is fluidly coupled with the peripheral lumen 134c. While only three peripheral lumen ports 132 are called out, it will be appreciated that depending on the length of the elongate shaft 112, and the number and relative positioning of the ultrasound transducers 56 within the treatment core 50, the elongate shaft 112 may include a greater number of peripheral lumen ports 134. It will be appreciated that the relative axial spacing of the peripheral lumen ports 134 may be determined based upon the relative spacing between adjacent ultrasound transducers 56, for example.

Therapeutic agents such as lysing drugs may be eluted into the vessel proximate each of the ultrasound transducers 56, for example. In some instances, at least some of the peripheral lumen ports 134 may be axially spaced from where the ultrasound transducers 56 will be positioned when the treatment core 50 is disposed within the central or inner lumen 128, particularly if there is a desire to elute at least some of the therapeutic agents upstream of where the ultrasound transducers 56 are located.

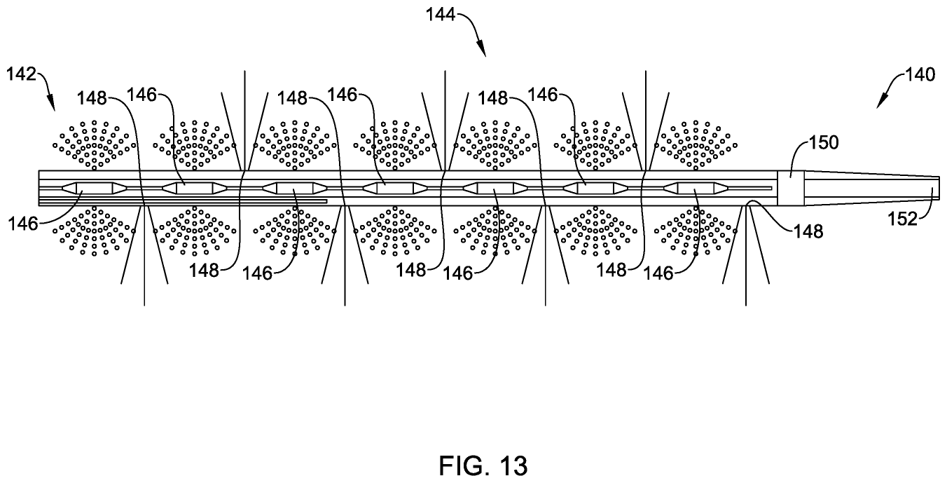

In some instances, the central lumen ports 132 may be positioned to elute cooling fluid adjacent to the ultrasound transducers 56 and the peripheral lumen ports 134 may be positioned to elute therapeutic agents adjacent to the ultrasound transducers 56. FIG. 13 is a schematic view of an illustrative system 140 in which an ultrasound catheter 142 is shown disposed within an elongate catheter 144. The ultrasound catheter 142 may be considered as being an example of the treatment core 50. The elongate catheter 144, which is shown partially transparent in order to be able to view the ultrasound catheter 142 therein, may be considered as being an example of the catheter 10 or the catheter 110.

The ultrasound catheter 142 includes a number of ultrasound transducers 146, each of which can be seen as creating ultrasonic energy that extends outwardly from each of the ultrasound transducers 146. While elution of therapeutic agents such as lysing drugs is not shown in FIG. 13, the elution of cooling media is. The elongate catheter 144 includes a number of cooling media ports 148, shown in this example as being positioned in between adjacent ultrasound transducers 146. The elongate catheter 144 includes a marker band 150 that may be used in ascertaining location of the system 140 relative to the vasculature and a desired treatment location within the vasculature, for example.

As will be appreciated, cooling media may be eluted into the vasculature near where each of the ultrasound transducers 146 are located. In some cases, the elongate catheter 144 may include a terminal cooling media port 152. In some instances, a physician or other professional may desire that some of the cooling media be eluted through the cooling media ports 148 extending through a side wall of the elongate catheter 144 and that some of the cooling media be eluted through the terminal cooling media port 152 located at a distal end of the elongate catheter 144.

In some instances, the physician or other professional may desire that all or substantially all (perhaps all but ten percent or less) of the cooling media exit the cooling media ports 148 and that none or very little of the cooling media exit the terminal cooling media port 152. In some instances, the physician or other professional may desire that all or substantially all (perhaps all but ten percent or less) of the cooling media exit the terminal cooling media port 152 and that none or very little of the cooling media exit the cooling media ports 148.

In some cases, the system 140 may be adapted to allow the physician or other professional to preferentially direct where cooling media exits the elongate catheter 144. As an example, a distal end of the ultrasound catheter 142 may be adapted to fit into and plug a distal end of the elongate catheter 144. By translating the ultrasound catheter 142 relative to the elongate catheter 144, the physician or other professional is able to block fluid from flowing through the terminal cooling media port 152 or to enable fluid to flow through the terminal cooling media port 152. Blocking the terminal cooling media port 152 will divert cooling media flow to elute through the cooling media ports 148. Opening the terminal cooling media port 152 will cause relatively less cooling media to elute through the cooling media ports 148.

As another example, a sheath (not shown) may be disposed over an exterior of the elongate catheter 144. The sheath may be adapted to block the cooling media ports 148, thereby causing all or substantially all of the cooling media to elute through the terminal cooling media port 152. Withdrawing the sheath proximally may cause the cooling media ports 148 to become unblocked, which will allow cooling media to elute through the cooling media ports 148, meaning that relatively less cooling media will elute through the terminal cooling media port 152. In some cases, the sheath may include elongate slots that correspond to where drug elution ports are located.

As a result, the sheath may be adapted to preferentially block the flow of cooling media through the cooling media ports while not blocking the flow of therapeutic agents such as lysing drugs through the drug elution ports.

In some instances, these ideas may be combined. The ultrasound catheter 142 may be translated relative to the elongate catheter 144 in order to open or effectively close the terminal cooling media port 152. In combination, a sheath may be advanced (or withdrawn) relative to the elongate catheter 144 in order to selectively block or open the cooling media ports 148.

As another example, it is contemplated that the elongate catheter 144 may be adapted to have separate cooling fluid lumens that can be independently connected to a source of cooling media, with a cooling fluid lumen fluidly coupled with the terminal cooling media port 152 (but not the cooling media ports 148) and one or more other cooling fluid lumens fluidly coupled with the cooling media ports 148 (but not the terminal cooling media port 152).

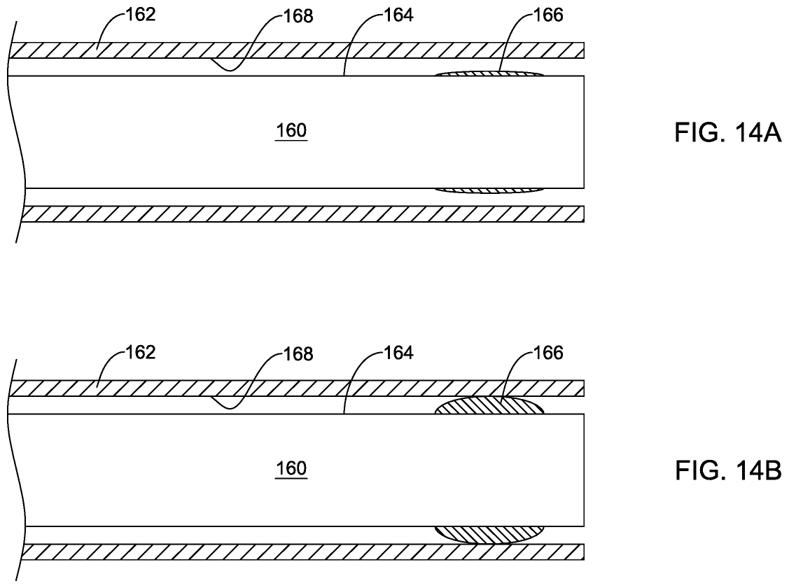

FIGS. 14A and 14B are schematic views of an illustrative example for selectively closing a terminal cooling media port such as the terminal cooling media port 152. In FIG. 14A, an ultrasound catheter 160 is shown disposed within an outer shaft 162. The ultrasound catheter 160 includes an outer surface 164. An inflatable balloon 166 is shown disposed on the outer surface 164. In FIG. 14A, the inflatable balloon 166 is shown as deflated, and as such does not prevent fluid flow between the outer surface 164 and an inner surface 168 of the outer shaft 162.

In FIG. 14B, the inflatable balloon 166 has been inflated sufficiently to contact the inner surface 168 of the outer shaft 162, thereby preventing fluid flow distal of the inflatable balloon 166. An inflation lumen (not shown) may extend through the ultrasound catheter 160 and may be fluidly coupled with an interior of the inflatable balloon 166. By selectively inflating or deflating the inflatable balloon 166, a physician or other professional are able to selectively allow the flow of cooling media out a distal end of the outer shaft 162.

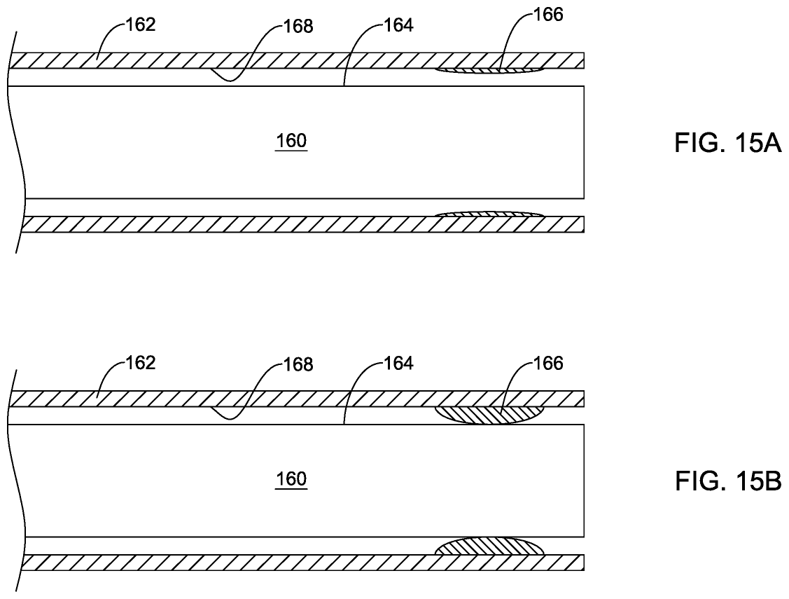

FIGS. 15A and 15B are schematic views of an illustrative example for selectively closing a terminal cooling media port such as the terminal cooling media port 152. In FIG. 15A, the ultrasound catheter 160 is shown disposed within the outer shaft 162. The ultrasound catheter 160 includes the outer surface 164 and the outer shaft 162 includes the inner surface 168. The inflatable balloon 166 is shown disposed on the inner surface 168. In FIG. 15A, the inflatable balloon 166 is shown as deflated, and as such does not prevent fluid flow between the outer surface 164 and the inner surface 168 of the outer shaft 162. In FIG. 15B, the inflatable balloon 166 has been inflated sufficiently to contact the outer surface 164 of the ultrasound catheter 160, thereby preventing fluid flow distal of the inflatable balloon 166. An inflation lumen (not shown) may extend through the elongate shaft 162. As an example, one of the peripheral lumens shown with respect to FIGS. 6-12 may be utilized as an inflation lumen and may be fluidly coupled with an interior of the inflatable balloon 166. By selectively inflating or deflating the inflatable balloon 166, a physician or other professional are able to selectively allow the flow of cooling media out a distal end of the outer shaft 162.

The materials that can be used for the various components of the devices described herein may include those commonly associated with medical devices. The devices and components thereof described herein may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), high-density polyethylene, low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of the devices described herein may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the devices described herein in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the devices described herein to achieve the same result.

In some embodiments, a degree of Magnetic Resonance Imaging (MRI) compatibility is imparted into the devices described herein. For example, the devices described herein, or portions thereof, may be made of a material that does not substantially distort the image and create substantial artifacts (e.g., gaps in the image). Certain ferromagnetic materials, for example, may not be suitable because they may create artifacts in an MRI image. The devices described herein, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative.

Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A system for treating a vascular region, the system comprising:
an elongate catheter shaft having a proximal region and a distal region, the elongate catheter shaft including:
an inner lumen extending through the elongate catheter shaft, the inner lumen adapted to allow a cooling media to pass therethrough;
a plurality of cooling media ports in fluid communication with the inner lumen;
a drug delivery lumen extending adjacent to the inner lumen; and
a plurality of drug delivery ports in fluid communication with the drug delivery lumen; and
an ultrasound catheter adapted to be disposed within the inner lumen, the ultrasound catheter including a plurality of ultrasound transducers.

2. The system of claim 1, wherein the inner lumen is adapted to allow for the cooling media to pass therethrough when the ultrasound catheter is disposed within the inner lumen.

3. The system of claim 1, wherein at least some of the plurality of cooling media ports are disposed within the distal region of the elongate catheter shaft.

4. The system of claim 3, wherein at least some of the plurality of cooling media ports extend through a side wall of the elongate catheter shaft.

5. The system of claim 1, wherein at least some of the plurality of cooling media ports are axially spaced from neighboring cooling media ports.

6. The system of claim 1, wherein at least some of the plurality of cooling media ports axially align relative to at least some of the plurality of ultrasound transducers when the ultrasound catheter is disposed within the inner lumen.

7. The system of claim 1, wherein at least some of the plurality of cooling media ports are arranged in a spiral configuration in which each cooling media port is axially spaced and circumferentially spaced from neighboring cooling media ports.

8. The system of claim 1, wherein at least some of the plurality of drug delivery ports are disposed within the distal region of the elongate catheter shaft.

9. The system of claim 1, wherein at least some of the plurality of drug delivery ports extend through a side wall of the elongate catheter shaft.

10. The system of claim 1, wherein at least some of the plurality of drug delivery ports are arranged in a spiral configuration in which each drug delivery port is axially spaced and circumferentially spaced from neighboring drug delivery ports.

11. The system of claim 1, wherein each drug delivery port is axially and/or circumferentially spaced from neighboring cooling media ports.

12. The system of claim 1, further comprising a terminal cooling media port disposed at a distal end of the elongate catheter shaft, the elongate catheter shaft and the ultrasound catheter adapted to selectively open or close the terminal cooling media port.

13. A system for treating a vascular region, the system comprising:
an ultrasound catheter including a plurality of ultrasound transducers;
an elongate catheter shaft defining a central lumen and a plurality of peripheral lumens extending through the elongate catheter shaft, the central lumen adapted to accommodate the ultrasound catheter extending therethrough, the elongate catheter shaft including a side wall;
a plurality of central lumen ports extending through the side wall and in fluid communication with the central lumen;
a plurality of peripheral lumen ports extending through the side wall and in fluid communication with the plurality of peripheral lumens.

14. The system of claim 13, wherein at least some of the plurality of central lumen ports are axially spaced from neighboring central lumen ports.

15. The system of claim 13, wherein at least some of the plurality of central lumen ports axially align relative to at least some of the plurality of ultrasound transducers when the ultrasound catheter is disposed within the central lumen.

16. The system of claim 13, wherein at least some of the plurality of central lumen ports are arranged in a spiral configuration in which each central lumen port is axially spaced and circumferentially spaced from neighboring central lumen ports.

17. The system of claim 13, wherein at least some of the plurality of peripheral lumen ports are arranged in a spiral configuration in which each peripheral lumen port is axially spaced and circumferentially spaced from neighboring peripheral lumen ports.

18. The system of claim 13, wherein each central lumen port is axially and/or circumferentially spaced from neighboring peripheral lumen ports.

19. A system for treating a vascular region, the system comprising:
an ultrasound catheter including a plurality of ultrasound transducers;
an elongate catheter shaft defining an inner lumen and a plurality of outer lumens extending through the elongate catheter shaft, the inner lumen adapted to accommodate the ultrasound catheter extending therethrough, the elongate catheter shaft including a treatment zone that corresponds to where the plurality of ultrasound transducers are when the ultrasound catheter is disposed within the inner lumen;
a plurality of inner lumen ports extending through the side wall and in fluid communication with the inner lumen; and
a plurality of outer lumen ports extending through the side wall and in fluid communication with the plurality of outer lumens.

20. The system of claim 19, wherein at least some of the plurality of inner lumen ports and at least some of the plurality of outer lumen ports are disposed within the treatment zone.
